(19) United States
(12) Patent Application Publication
(10) Pub. No.: US 2022/0177399 A1
(43) Pub. Date: Jun. 9, 2022

(54) SYSTEMS AND METHODS FOR GENERATING A CARBOXYLIC ACID FROM A CO2 GAS STREAM

(71) Applicant: SHELL OIL COMPANY, HOUSTON, TX (US)

(72) Inventors: Andreas KLEMT, Leuna (DE); Sven SCHOLZ, Leuna (DE)

(21) Appl. No.: 17/523,161

(22) Filed: Nov. 10, 2021

(30) Foreign Application Priority Data
Nov. 10, 2020 (EP) 20206713.8

(51) Int. Cl. C07C 51/02; C01D 7/07; C07C 51/41; C25B 1/04; C25B 1/26

(52) U.S. Cl. CPC C07C 51/02; C01D 7/07; C25B 1/26; C25B 1/04; C07C 51/41

(57) ABSTRACT

A method for generating a carboxylic acid from carbon dioxide (CO₂), the method includes (a) feeding a gas stream having the CO₂ to a first reactor having a base (MOH) to produce bicarbonate (MHCO₃) and (b) feeding the MHCO₃ generated in the first reactor to a second reactor disposed downstream from the first reactor. The second reactor includes a catalyst. The method also includes (c) contacting the MHCO₃ with hydrogen gas in the presence of the catalyst in the second reactor to produce formate (HCOOM) and (d) electrolysing an aqueous solution of a metal halide (MCl) in a chloro-alkali electrolysis reactor fluidly coupled to the first reactor, the second reactor, or both to produce at least a portion of the MOH, the hydrogen gas and Cl₂. The portion of the MOH is used in step (a) and the carboxylic acid is formic acid (HCOOH).

### REFERENCE TO RELATED APPLICATION

The present application claims the benefit from the priority of European Patent Application No. 20206713, entitled “SYSTEMS AND METHODS FOR GENERATING A FORMIC ACID PRECURSOR FROM A CO₂ GAS STREAM,” filed Nov. 10, 2020, and is hereby incorporated by reference in its entirety.

### FIELD OF THE DISCLOSURE

The present disclosure relates, in some embodiments, to systems and methods that convert CO₂ (e.g., from a CO₂ stream produced by an oil and gas industry asset) into a viable commercial product. In particular, the system and methods convert CO₂ into formic acid precursors (e.g., HCOOK, HCOONa) and/or formic acid (HCOOH).

### BACKGROUND OF THE DISCLOSURE

A large portion of the energy used by consumers is derived from the processing and combustion of fossil fuels (e.g., hydrocarbon fuels). However, besides generating energy, these combustion processes may also generate undesirable greenhouse gases (e.g., carbon dioxide (CO₂)).

Two main strategies are used in controlling anthropogenic emissions of greenhouse gases. The first strategy is to simply find ways to reduce the overall fossil fuel consumption through energy use limitation or use of alternative energy methods (e.g., solar, wind, electric).

However, this strategy does not mitigate the formation of the greenhouse gases. The second strategy includes abatement of generated greenhouse gases, for example, through their transformation into environmentally benign or even beneficial products. Greenhouse gas abatement technology allows for fossil fuel consumption without decreasing or restricting fossil fuel consumption.

Carbon dioxide (CO₂) is the primary gas in greenhouse gases. There are currently technologies that capture and store CO₂ (e.g., as it is generated from fossil fuel processing or combustion system). However, these technologies are limited to CO₂ gas abatement without providing additional commercial and/or environmental benefits.

### SUMMARY

In a first embodiment, a method for generating a carboxylic acid from carbon dioxide (CO₂), the method includes (a) feeding a gas stream having the CO₂ to a first reactor having a base (MOH) to produce bicarbonate (MHCO₃) and (b) feeding the MHCO₃ generated in the first reactor to a second reactor disposed downstream from the first reactor. The second reactor includes a catalyst. The method also includes (c) contacting the MHCO₃ with hydrogen gas in the presence of the catalyst in the second reactor to produce formate (HCOOM) and (d) electrolysing an aqueous solution of a metal halide (MCl) in a chloro-alkali electrolysis reactor fluidly coupled to the first reactor, the second reactor, or both to produce at least a portion of the MOH, the hydrogen gas and Cl₂. The portion of the MOH is used in step (a) and the carboxylic acid is formic acid (HCOOH).

In a second embodiment, a system for generating a carboxylic acid from carbon dioxide (CO₂) includes a first reactor fluidly coupled to a gas source having the CO₂ and that may combine the CO₂ with a base (MOH) to generate bicarbonate (MHCO₃) and, optionally, an off gas and a second reactor disposed downstream from and fluidly coupled to the first reactor and having a catalyst. The second reactor may receive the bicarbonate and hydrogen gas and produce formate (HCOOM), and a temperature and hydrogen pressure within the second reactor is in the range of from 15° C. to 210° C. and from 0.001 bara to 100 bara, respectively. The system also includes a chloro-alkali electrolysis reactor disposed downstream from and fluidly coupled to the first reactor and the second reactor. The chloro-alkali electrolysis reactor may produce at least a portion of the base, a hydrogen gas and chlorine (Cl₂), and to provide at least a portion of the base to the first reactor.

In a third embodiment, a method for generating a carboxylic acid from carbon dioxide (CO₂) includes (a) mixing a gas stream having the CO₂ with a base (MOH) to produce bicarbonate (MHCO₃), (b) contacting the MHCO₃ with hydrogen gas in the presence of the catalyst to produce formate (HCOOM), and (d) electrolysing an aqueous solution of a metal halide (MCl) to produce at least a portion of the MOH used in step (a). The carboxylic acid is formic acid (HCOOH).

### BRIEF DESCRIPTION OF THE DRAWINGS

Some embodiments of the disclosure may be understood by referring, in part, to the present disclosure and the accompanying drawings, in which:

FIG. 1 is a diagram of a system for generating formic acid (HCOOH) from a carbon dioxide (CO₂)-containing gas stream, whereby the system includes an ion exchange resin reactor, in accordance with an embodiment of the present disclosure;

FIG. 2 is a diagram of system for generating HCOOH from a CO₂-containing gas stream, whereby the system includes an esterification reactor, in accordance with an embodiment of the present disclosure;

FIG. 3 is a diagram of a system for generating HCOOH from a CO₂-containing gas stream, whereby the system includes a reactor that convers formate into the HCOOH, in accordance with an embodiment of the present disclosure; and

FIG. 4 is a plot of a concentration of formate and sodium at a column outlet in millimoles (mmol)/liter (L) as a function of elution volume in milliliters (mL), in accordance with an embodiment of the disclosure.

### DETAILED DESCRIPTION

One or more specific embodiments of the present disclosure will be described below. These described embodiments are examples of the presently disclosed techniques. Additionally, in an effort to provide a concise description of these embodiments, not all features of an actual implementation may be described in the specification. It should be appreciated that in the development of any such actual implementation, as in any engineering or design project, numerous implementation-specific decisions will be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

The terms “approximately,” “about,” and “substantially” as used herein represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of a stated amount.

Existing techniques for CO₂ gas abatement do not convert CO₂ into commercially viable products nor provide an environmental benefit. Accordingly, there is a need for improved methods and systems for CO₂ gas abatement which also provide commercial and/or environmental benefits. The present disclosure describes methods and systems for transforming a CO₂ gas stream (e.g., derived from fossil fuel processing or combustion system) into formic acid (HCOOH), a multi-functional base chemical (e.g., a preservative and an antibacterial agent in livestock feed, a fuel for electric cars having formic acid fuel cells, a hydrogen storage material for hydrogen fuel cells, an additive for various cleaning products, an intermediary to produce isobutanol from CO₂ using microbes, a de-icer) and its precursors.

Disclosed herein are systems and methods for generating a formic acid precursor (HCOOM) and/or a formic acid (HCOOH) from a carbon dioxide (CO₂) gas stream. The disclosed system and method advantageously convert CO₂ into formic acid, a multi-functional base chemical that may be readily commercialized and precursors thereof (e.g., HCOOK, HCOONa). Formic acid may desirably be used, for example, as a preservative and an antibacterial agent in livestock feed, a fuel for electric cars having formic acid fuel cells and indirectly in hydrogen fuel cells as a hydrogen storage material, an additive for various cleaning products, an intermediary to produce isobutanol from CO₂ using microbes, a de-icer, and many other uses. Additionally, as disclosed herein, the system and method may directly convert diluted CO₂ streams into formic acid in a scalable and less complex manner in comparison to existing chemical synthetic technologies that may produce undesirable waste products. Additionally, many of the method steps and system components disclosed herein involve catalytic cycles that recycle by-products from other methods steps and system components, thereby minimizing waste-products.

In particular, the embodiments disclosed herein provide a first process step (a) in which a gas stream having CO₂ is mixed with base (MOH) such that the CO₂ in the gas stream reacts with the alcohol to produce MHCO₃ and a second process step (b) that includes combining the MHCO₃ produced in step (a) with hydrogen gas (H₂) in the presence of a catalyst to produce HCOOM. Additional embodiments include electrolysing an aqueous solution of MCl in, for example, a chloro-alkali electrolysis process to produce at least a MOH, hydrogen gas and a chlorine (Cl₂), and using the MOH produced from the electrolysis process in step (a). Therefore, the CO₂ contained in the gas stream may be combined with the MOH under certain reactor conditions to form a formic acid precursor HCOOM. The disclosed process includes an intermediate process step that generates an intermediate MHCO₃ from the CO₂ and MOH and converts the MHCO₃ into the formic acid precursor HCOOM.

### Systems for Converting CO₂ to Formic Acid

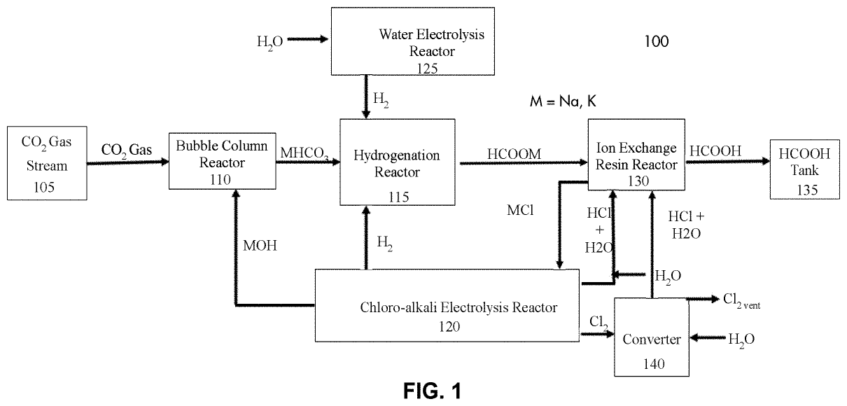

With the foregoing in mind, FIG. 1 is a block diagram of a system 100 for converting a CO₂ to formic acid or other water-soluble carboxylic acid (e.g., oxalic acid). The following description, the system components are discussed in the context of being connected and fluidly coupled to on another. However, it should be appreciated that one or more the system components disclosed herein may be separate from one another and located at a remote location without departing from the scope of the present disclosure. As such, the fluids generated by the system components located in remote locations may be transferred (e.g., via a transfer vehicle) to a different location for further processing to produce the formic acid. The system 100 includes a bubble column reactor 110 connected and fluidly coupled to a CO₂ gas stream 105 through a CO₂ gas transfer line. The CO₂ gas stream 105 may have between approximately 20 to 100% vol. % CO₂. For example, the CO₂ gas stream 105 may have between 50 to 100 vol. %, 8- to 100 vol. %, 20 to 50 vol. %, 80 vol. % or 100 vol. % CO₂. In the illustrated embodiment, the bubble column reactor 110 is fluidly coupled to a hydrogenation reactor 115 (e.g., a trickle bed/liquid phase reactor) through a bicarbonate (MHCO₃) transfer line, and to a chlorine-alkali electrolysis reactor 120 through a base (MOH) transfer line. By way of non-limiting example, the bicarbonate transfer line provides potassium bicarbonate (KHCO₃) and/or sodium bicarbonate (NaHCO₃) from the bubble column reactor 110 to the hydrogenation reactor 115, and the base transfer line provides potassium hydroxide (KOH) and/or sodium hydroxide (NaOH) from the chloro-alkali electrolysis reactor 120 to the bubble column reactor 110. The hydrogenation reactor 115 may be a fixed bed reactor, fluidized bed reactor, or any other suitable reactor that hydrogenates bicarbonate to form a hydrogenated product having formate (HCOOM). In addition to the formate, the hydrogenated product may include unreacted CO₂, bicarbonate, or both. For example, the hydrogenated product may be a mixture of from between 1-99 wt. % formate, 1-99 wt. % bicarbonate, and 1 to 99 wt. % CO₂ based on a total weight of the hydrogenated product. In certain embodiments, the mixture may have an amount of formate that is between 10 to 90 wt. % and preferably between 40 to 60 wt. %, between 10 to 90 wt. %, and preferably between 40 to 60 wt. % of MHCO₃, and between 10 to 90 wt. %, and preferably between 40 to 60 wt. % of CO₂ based on the total weight of the hydrogenated product. In certain embodiments, the mixture has substantially no detectable CO₂. For example, in one embodiment, the hydrogenated product includes a mixture of formate and bicarbonate. However, in other embodiments, the hydrogenated product consists essentially of formate.

In the illustrated embodiment, the hydrogenation reactor 115 is also connected and fluidly coupled to the chloro-alkali electrolysis reactor 120 through a first hydrogen (H₂) gas line, a water electrolysis reactor 125 through a second H₂ gas line, and an ion exchange resin reactor 130 through a formate (HCOOM) transfer line. In certain embodiments, the hydrogenation reactor 115 feeds formate to a reactor disposed between the hydrogenation reactor 115 and the ion exchange resin reactor 130. The reactor (e.g., a furnace) may convert the formate into oxalate (MOOC-COOM), which is converted to oxalic acid in the ion exchange resin reactor 130. For example, the formate may be heated in the reactor to a temperature between about 250° C. to 500° C. to deprotonate the formate, thereby forming oxalate and H₂.

As discussed above, the system 100 includes the ion exchange resin reactor 130 positioned downstream of and fluidly coupled to the hydrogenation reactor 115. The ion exchange resin reactor 130 is connected and fluidly coupled to a formate (HCOOH) tank 135 through a formic acid (HCOOH) transfer line, to a converter 140 through a hydrochloric acid (HCl) transfer line, and to the chloro-alkali electrolysis reactor 120 through a metal halide (MCl) transfer line. The ion exchange resin reactor 130 may include a protonated ion exchange resin. In operation, the ion exchange resin reactor 130 protonates the formate to generate formic acid (HCOOH) and, optionally, the metal halide (e.g., sodium chloride (NaCl) and/or potassium chloride (KCl)).

The metal halide transfer line may provide KCl, NaCl, or both to the chloro-alkali electrolysis reactor 120. Optionally, in certain embodiments, the MCl transfer line feeds a metal halide solution, such as KCl or NaCl, obtained from the ion exchange resin reactor 130 and feeds the MCl solution to a salt purifier. The salt purifier generates a purified salt that is provided to the chloro-alkali electrolysis reactor 120. The purified MCl generated in the salt purifier may be in solution.

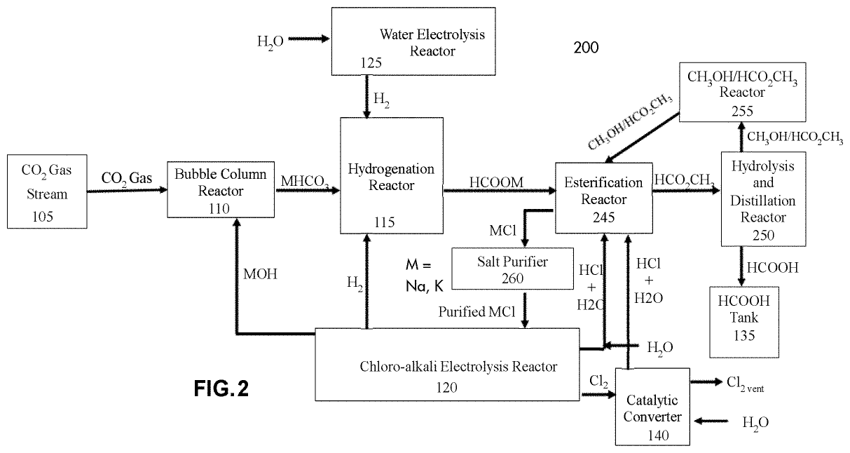

In some embodiments, the MCl solution may be generated by an esterification rector instead of the ion exchange resin reactor 130. For example, FIG. 2 is an embodiment of a system 200 having an esterification reactor 245 connected and fluidly coupled to the hydrogenation reactor 115 through the HCOOM transfer line, the chloro-alkali electrolysis reactor 120 through the MCl transfer line, the converter 140 through the HCl transfer line, a hydrolysis and distillation reactor 250 through a methyl formate (HCO₂CH₃) transfer line, and to a methanol/methyl formate (CH₃OH/HCO₂CH₃) reactor 255 through a methanol/methyl formate (CH₃OH/HCO₂CH₃) transfer line. In operation, the esterification reactor 245 first protonates the formate in the hydrogenated product using a mono-alcohol (e.g., methanol (CH₃OH) and/or ethanol (CH₃CH₂OH)) in the presence of HCl to generate an ester and the metal halide (e.g., NaCl and/or KCl). In certain embodiments, the formate generated in the hydrogenation reactor 115 is fed to a reactor disposed between the hydrogenation reactor 115 and the esterification reactor 245. The reactor (e.g., a furnace) may convert the formate into oxalate (MOOC-COOM). For example, the formate may be heated in the reactor to a temperature between about 250° C. to 500° C. to deprotonate the formate, thereby forming oxalate and H₂. The oxalate is fed to the esterification reactor 245 along with HCl and an alcohol (e.g., methanol or ethanol) to convert the oxalate to oxalic acid ester (e.g., oxalic acid dimethyl ester or oxalic acid diethyl ester). The oxalic acid ester is further processed to generated oxalic acid.

The system 200 also includes a hydrolysis and distillation reactor 250 disposed downstream from and fluidly coupled to the esterification reactor and upstream from and fluidly coupled to the HCOOH tank 135 through the HCOOH transfer line, and to the CH₃OH/HCO₂CH₃ reactor 255 reactor through the CH₃OH/HCO₂CH₃ transfer line. In operation, the hydrolysis and distillation reactor 250 receives the ester from the esterification reactor 245 and protonates the ester, in a second protonation step, to generate the formic acid.

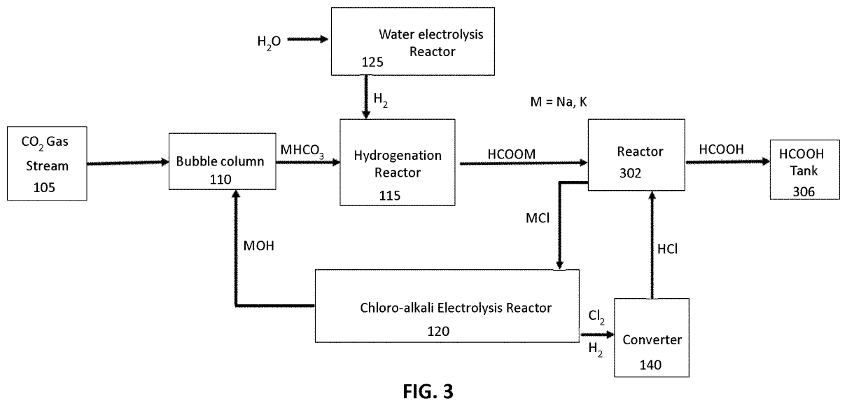

FIG. 3 illustrates another embodiment for generating formic acid or oxalic acid from CO₂ in accordance with present disclosure. For example, FIG. 3 is an embodiment of a system 300 that may be used to convert CO₂ into formic acid or oxalic acid. The system 300 includes the bubble column reactor 110, the hydrogenation reactor 115, the water electrolysis reactor 125, and the chloro-alkali electrolysis reactor 120, and the converter 140. In addition, the system 300 includes a reactor 302 and a HCOOH tank 306. The reactor 302 may be a protonated cation exchange resin reactor, optionally protonated with HCl, a distillation reactor, or a two-stage reactor having an esterification reactor and a hydrosylation reactor. In operation, the reactor 302 receives the formate produced in the hydrogenation reactor 115 and converts the formate into a water-soluble carboxylic acid. In addition to the carboxylic acid, the reactor 302 generates the metal halide provided to the chloro-alkali electrolysis reactor 120. For example, the reactor 305 receives the formate and protonates it by ion exchange between the protonated cation exchange resin and the formate to produce formic acid. Alternatively, the formate may be combined with HCl (e.g., an HCl gas stream or aqueous HCl) in the reactor 302 (e.g., a distillation reactor) to generate the formic acid and MCl. In another embodiment, the formate may be protonated in a first step with a mono-alcohol in the presence of HCl in a first section of the reactor 302 having an esterification reactor, to produce an ester and MCl. Following the first protonation step, the ester may undergo a second protonating step in a second section of the reactor 302 having a hydrolysation reactor to hydrolyse the ester and generate the formic acid. By way of non-limiting example, the carboxylic acid is formic acid, oxalic acid, or both. The reactor 302 may feed the carboxylic acid to the HCOOH tank 306 for storage.

Returning to FIG. 1, the system 100 also includes a CO₂ gas stream 105, which may be provided by a tank or streamed from a CO₂ gas source such as a device containing a fuel combustion or processing component. The tank or CO₂ gas source is upstream from and fluidly coupled to the bubble column reactor 110 through a CO₂ gas transfer line that provides the CO₂ gas stream 105 to the bubble column reactor 110. Within the bubble column reactor 110, the CO₂ gas stream 105 may be combined with a base (MOH) such as, for example, aqueous potassium hydroxide (KOH) and/or aqueous sodium hydroxide (NaOH) to produce one or more bicarbonate compounds (MHCO₃). By way of non-limiting example, the MHCO₃ may be potassium bicarbonate (KHCO₃) and/or sodium bicarbonate (NaHCO₃) depending on the base used. The bubble column reactor 110 includes a bubble column having a vertically-arranged or horizontally arranged column of any suitable shape and size. In some embodiments, the CO₂ gas transfer line may provide a CO₂ gas to the bubble column at any location along the bubble column. For example, the CO₂ gas transfer line may provide the CO₂ gas at a bottom, top, or any other position between the bottom and the top of a bubble column. For example, the bubble column reactor 110 may receive the CO₂ gas from the CO₂ gas transfer line at a position of the bubble column found in the lower half of the bubble column.

As shown in FIG. 1, the system 100 includes the hydrogenation reactor 115. The hydrogenation reactor 115 includes a catalyst that facilitates formation of formate (HCOOM) from the bicarbonate compounds. By way of non-limiting example, the catalyst may be a palladium catalyst, a nickel catalyst, a platinum catalyst, copper catalyst, and combination thereof. The palladium catalyst may include Pd/carbon, and the nickel catalyst may include Ni/SiO₂. Examples of catalysts suitable for the hydrogenation of bicarbonate to formate include 0.1-5 wt. % Pd on carbon or theta alumina or titania; 10-70 wt. % Ni/SiO₂ or Ni/ASA—precipitated; 10-40 wt. % Ni/theta alumina—impregnated, copper wire, and any other suitable catalyst that facilitates hydrogenation of bicarbonate to form formate.

Typical liquid space velocities inside a fixed bed reactor are 0.1-5 volume feed/(volume catalyst*hour) (v/(vh)). Alternatively, a stirred tank reactor with suspended catalyst may be used, preferably with a catalyst concentration in the range of 0.01 g/L to 100 g/L.

In FIG. 1, the hydrogenation reactor 115 is connected and fluidly coupled to the ion exchange resin reactor 130 through the HCOOM transfer line and the chloro-alkali electrolysis reactor 120 through the first H₂ gas transfer line. In some embodiments, the hydrogenation reactor 115 may mix or otherwise combine the MHCO₃ (e.g., KHCO₃, NaHCO₃) with hydrogen gas provided by one or more water electrolysis reactors 125 and the chloro-alkali electrolysis reactor 120 at a temperature ranging from about 15° C. to about 75° C. and a hydrogen pressure ranging from about 0.001 bara to about 100 bara to produce formate (HCOOM) such as HCOOK, HCOONa, or both depending on the base provided to the bubble column reactor 110. The hydrogen gas provided to the hydrogenation reactor 115 may be at a temperature of about 15° C., or about 20° C., or about 25° C., or about 30° C., or about 35° C., or about 40° C., or about 45° C., or about 50° C., or about 55° C., or about 60° C., or about 65° C., or about 70° C., or about 75° C. According to some embodiments, the hydrogenation reactor 115 may include a hydrogen pressure of about 0.001 bara, or about 0.005 bara, or about 0.01 bara, or about 0.05 bara, or about 0.1 bara, or about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara. However, in certain embodiments, the hydrogen pressure may be more than about 1.0 bara such as, for example, about 10 bara, about 20 bara, about 30 bara, about 40 bara, about 50 bara, about 60 bara, about 70 bara, about 80 bara, about 90 bara, or about 100 bara, where about includes plus or minus 5 bara.

Once the HCOOM is produced in the hydrogenation reactor 115, it may be transferred to the ion exchange resin reactor 130 through the HCOOM transfer line and converted to HCOOH (i.e., formic acid). The ion exchange resin reactor 130 includes an ion exchange resin. The ion exchange resin includes a polymer that acts as a medium for ion exchange. The ion exchange resin may be a cation-exchange resin such as, for example, Dowex 50WX8 or any other suitable cation-exchange resin. The ion exchange resin may be strongly acidic or weakly acidic.

Following formation of the formic acid in the ion exchange resin reactor 130, it may be fed to the HCOOH tank 135 through the HCOOH transfer line for storage and/or distribution. The HCOOH tank 135 may be any suitable shape and size for containing the formic acid produced by the system 100, 200 and made of any material (e.g., a plastic, a glass, a metal) suitable for storing formic acid without affecting the integrity of the material. The HCOOH tank 135 may also include an apparatus for transferring the formic acid to another location. For example, the HCOOH tank 135 may connected to and fluidly coupled to a pipeline that directs the formic acid to a desired location. The HCOOH tank 135 may also be removably coupled (e.g., via a pipe or conduit) to a container on a vehicle (e.g., a transfer truck) used to transfer the formic acid to another location.

As discussed above, the system 100 includes the water electrolysis reactor 125 that is fluidly coupled to the hydrogenation reactor 115 through the second H₂ transfer line. The water electrolysis reactor 125 may split water into oxygen (O₂) and H₂ and transfer the H₂ to the hydrogenation reactor 115. The water electrolysis reactor 125 may include one or more electrodes, a container, a water input line, and an oxygen gas outlet. In operation, the water electrolysis reactor 125 may generate from about 1% to about 100% of the H₂ used by the hydrogenation reactor 115. For example, the water electrolysis reactor 125 may generate from about 1%, about 10%, about 20%, about 30%, about 40%, about 50%, about 60%, about 70%, about 80%, about 90%, or about 100% of the H₂ used by the hydrogenation reactor 115, where about includes plus or minus 5%.

In addition to receiving H₂ from the water electrolysis reactor 125, the hydrogenation reactor 115 may receive H₂ from the chloro-alkali electrolysis reactor 120, as shown in FIG. 1. In some embodiments, the chloro-alkali electrolysis reactor 120 includes one or more electrodes, one or more membranes for separating the electrodes, a container to contain other components of the chloro-alkali electrolysis reactor 120, a water inlet, and a salt inlet. As shown in FIG. 1, the chloro-alkali electrolysis reactor 120 receives the MCl (e.g., KCl and/or NaCl) from the ion exchange resin reactor 130 through the MCl transfer line. Once received, the chloro-alkali electrolysis reactor 120 may convert, in the presence of water, the MCl into H₂, Cl₂, and MOH, where M is sodium (Na) or potassium (K). In certain embodiments, the MCl is concentrated prior to use by evaporating water. In operation, the chloro-alkali electrolysis reactor 120 may generate from about 1% to about 100% of the H₂ used by the hydrogenation reactor 115. For example, the chloro-alkali electrolysis reactor 120 may generate from about 1%, about 10%, about 20%, about 30%, about 40%, about 50%, about 60%, about 70%, about 80%, about 90%, or about 100% of the H₂ used by the hydrogenation reactor 115, where about includes plus or minus 5%. In some embodiments, the chloro-alkali electrolysis reactor 120 is mercury free, which is an advantage over existing technologies requiring mercury. That is, mercury is not used as an additive in this particular embodiment. By-products (e.g., hypochlorite (MOCl) and/or chlorate (MClO₃)) generated in the chloro-alkali electrolysis reactor 120 may be removed in a cleaning step.

As discussed above, the system 100 also includes the converter 140, which is connected and fluidly coupled to the chloro-alkali electrolysis reactor 120 via a chlorine (Cl₂) transfer line through which it may receive Cl₂ generated by the chloro-alkali electrolysis reactor 120. The converter 140 may mix or otherwise combine the Cl₂ with water to form HCl. In this step, the Cl₂ may be mixed with water and contacted with a bed of carbon to generate an HCl containing stream which may be fed to the ion exchange resin reactor 130 through the HCl transfer line. In certain embodiments, at least 1 to 100 vol. % of the H₂ and Cl₂ generated in the chloro-alkali electrolysis reactor 120 may be combined with water in the convertor 140. For example, between from 1 to 99 vo. %, preferably between from 90 to 100 vol. %, of the H₂ and Cl₂ are combined with water in the convertor 140 to generate HCl. In one embodiment, between from 75 to 90 vol. % of the Cl₂ from the chloro-alkali electrolysis reactor 120 is combined with the water in the convertor 140. In other embodiments, at least a portion of the H₂ combined with the water in the convertor 140 is provided by the water electrolysis reactor 125. For example, between from 1 to 99 vol. %, preferably 1 to 50 vol. %, 1 to 15 vol. %, or 1 to 15 vol. % of the H₂ from the water electrolysis reactor 125 is provided to the convertor 140. In certain embodiment, 100 vol % of the H₂ combined with the water in the convertor 140 is from the water electrolysis reactor 125. That is, in this particular embodiment, substantially no H₂ from the chloro-alkali electrolysis reactor 120 is provided to the convertor 140 and mixed with the water. The convertor 140 may be a catalytic convertor, a thermal convertor, or any other suitable system that converts H₂ and Cl₂ into HCl. At least a portion of the HCl generated in the convertor 140 may be used for protonating the formate to produce the carboxylic acid (e.g., formic acid, acetic acid, and/or oxalic acid) in the ion exchange resin reactor 130, the esterification reactor 245 (FIG. 2), or reactor 302 (FIG. 3).

As discussed above, with reference to FIG. 2, the system 200 include the esterification reactor 245 instead of the ion exchange resin reactor 130. In this particular embodiment, esterification may be carried out at acidic conditions (e.g., at a pH<7). The esterification reactor 245 may receive the HCOOM from the hydrogenation reactor 115 and combine it with HCl from the catalytic converter 140 and methanol (CH₃OH) received from the CH₃OH/HCO₂CH₃ reactor 255 to produce methyl formate (HCO₂CH₃). In certain embodiment, the reactor 255 may provide ethanol (CH³CH₂OH) instead of methanol to the esterification reactor 245 to produce ethyl formate (HC₂CH₂CH₃). Once the HCO₂CH₃ is generated, it may be fed to the hydrolysis and distillation reactor 250 which reacts the HCO₂CH₃ with water at a temperature ranging from about 25° C. to about 100° C. to form formic acid (HCOOH). The formic acid may be collected in the HCOOH tank 135. In some embodiments, the hydrolysis and distillation reactor 250 may combine the HCO₂CH₃ and water at a temperature of about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., or about 100° C., where about includes plus or minus 2.5° C. The hydrolysis and distillation reactor 250 may include a heating source (e.g., heating element), a reaction tank, and a pressure regulator.

### Methods for Converting a CO₂ to a Formic Acid

Embodiments of the present disclosure also include a method for generating formic acid from a CO₂ gas stream. An exemplary pathway for generating formic acid (HCOOH) in accordance with the present disclosure includes the stoichiometry shown below:

| | | |
|---|---|---|
| 1) | 2 KOH + 2 CO₂ | → 2 KHCO₃ |
| 2) | 2 KHCO₃ + 2 H₂ | → 2 KOOCH + 2 H₂O |
| 3a) | 2 KOOCH + 2 H-IE | → 2 KOOCH + 2 K-IE |
| 3b) | 2 HCl + 2 K-IE | → 2 KCl + 2 H-IE |
| 4) | 2 KCl + 2 H₂O | → Cl₂ + H₂ + 2 KOH |
| 5) | Cl₂ + H₂O | → 2 HCl + ½ O₂ |
| 6) | H₂O | → H₂ + ½ O₂ |
| SUM | 2 CO₂ + 2 H₂O | → 2 HOOCH + O₂ |

The disclosed method includes using t bubble column reactor (e.g., the bubble column reactor 110) to convert CO₂ in the CO₂ gas stream into a bicarbonate compound (MHCO₃) such as, for example, KHCO₃ and/or NaHCO₃ depending on the base used, and a hydrogenation reactor (e.g., the hydrogenation reactor 115) to convert the MHCO₃ to the respective metal formate (HCOOM, where M is either K or Na), an esterification reactor (e.g., the esterification reactor 245) to convert the HCOOM to methyl format (HCO₂CH₃), and a hydrolysis and distillation reactor (e.g., the hydrolysis and distillation reactor 250) to convert the HCO₂CH₃ to formic acid.

Preferably, the stoichiometry of the pathway for generating formic acid via the hydrolysis and distillation reactor is shown below:

| | | |
|---|---|---|
| 1) | HOOCH + CH₃OH | → HCOOCH₃ ↑ + H₂O |
| 2) | destillation | |
| 3) | HCOOCH₃ + H₂O | → HOOCH + CH₃OH ↑ |
| 4) | hydrolysis | |

Alternatively, in accordance with an embodiment of the disclosed method, the metal formate (HCOOM, where M is Na or K) may be combined with HCl (e.g., HCl gas stream or aqueous HCl) in a distillation reactor to form formic acid according to the stoichiometry pathway shown below:

| | | |
|---|---|---|
| 1) | 2 KOH + 2 CO₂ | → 2 KHCO₃ |
| 2) | 2 KHCO₃ + 2 H₂ | → 2 KOOCH + 2 H₂O |
| 3) | 2 KOOCH + 2 HCl | → 2 HOOCH + 2 KCl |
| 4) | 2 KCl + 2 H₂O | → Cl₂ + H₂ + 2 KOH |
| 5) | Cl2 + H₂O | → 2 HCl + ½ O₂ |
| 6) | H₂O | → H₂ + ½ O₂ |
| SUM | 2 CO₂ + 2 H₂O | → 2 HOOCH + O₂ |

As discussed above, the method presented herein may include passing a CO₂ gas stream having CO₂ through a reactor (e.g., the bubble column reactor 110) having a base (e.g., MOH where M is Na or K) to produce the MHCO₃ and an off gas. According to some embodiments, the CO₂ gas may be converted to MHCO₃ in a separated unit outside of system (e.g., the system 100, 200, 300) instead of in the bubble column reactor. In this particular embodiment, the MHCO₃ is generated in a remote location and transferred to the system, for example, via a transfer vehicle. The CO₂ gas stream may be from about 0.01 vol % CO₂ to about 100 vol % CO₂ (vol % relative to the total gas stream volume). The disclosed method may include a step of combining the MHCO₃ produced by the bubble column reactor with hydrogen gas at a temperature up to 210° C. in the hydrogenation reactor to form HCOOM, where M is Na or K. For example, the temperature may range from about 12.5° C. to about 210° C., from about 15° C. to about 200° C., from about 15 to about 150° C., from about 15° C. to about 75° C. In certain embodiments, the hydrogen gas may be at a temperature of about 15° C., about 20° C., about 25° C., about 30° C., about 35° C., about 40° C., about 45° C., about 50° C., about 55° C., about 60° C., about 65° C., about 70° C., or about 75° C., where about includes plus or minus 2.5° C.

In other embodiments, the method includes a step of combining the MHCO₃ (e.g., KHCO₃ and/or NaHCO₃) produced by the bubble column reactor with a catalyst and hydrogen gas in the hydrogenation reactor (e.g., the hydrogenation reactor 115) to produce formate (HCOOM) at a temperature ranging from 15° C. to 210° C. and a hydrogen pressure ranging from about 0.001 bara to about 100 bara for 6 to 24 h. The hydrogen pressure may be about 0.001 bara, about 0.005 bara, about 0.01 bara, about 0.05 bara, about 0.1 bara, about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara. The hydrogen pressure may be about 1.0 bara, about 10 bara, about 20 bara, about 30 bara, about 40 bara, about 50 bara, about 60 bara, about 70 bara, about 80 bara, about 90 bara, or about 100 bara, where about includes plus or minus 5 bara.

The hydrogenation reactor may be a fixed bed reactor having liquid space velocities in the range of from about 0.1 to 5 h⁻¹. For example, the hydrogenation reactor may be a trickle-bed or liquid phase reactor. In certain embodiments, the hydrogenation reactor may be a tank reactor having a suspended catalyst. In one embodiment, the hydrogenation reactor may be a loop reactor and include one or more catalysts. By way of non-limiting example, the catalyst in the hydrogenation reactor includes a copper catalyst, a palladium catalyst, a nickel catalyst, and combinations thereof. Catalysts containing copper or nickel may be in the form of a wire, powder, shavings, granules, and combinations thereof of respective metal. The catalyst may be in an amount of from about 0.001 to 70.0 wt. % in relation to the carrier material. For example, the catalyst may be in an amount of from 0.01 to 40.0 wt. %, 10.0 to 70.0 wt. %, 10.0 to 40.0 wt. %, 0.1 to 5.0 wt. %, 0.01 wt. %, 5.0 wt. %, 10.0 wt. %, 40.0 wt. %, or 70.0 wt. % each in relation to the carrier material. The palladium catalyst may include Pd/Al₂O₃, 0.1-5 wt. % (based on carrier material) Pd on carbon or theta alumina or titania; 10-70 wt. % (based on carrier material) Ni/SiO₂ or Ni/ASA (amorphous silica alumina)—precipitated; and 10-40 wt. % (based on carrier material) Ni/theta alumina—impregnated, and combinations thereof. In certain embodiments, the catalyst is a copper catalyst. For example, the copper catalyst may be a copper wire, copper powder, copper shavings, copper granules, and combinations thereof. The copper catalyst may or may not be supported on a carrier. When using the copper catalyst, Cu²⁺ ions may be produced after at least 100 h. For example, after at least between 5 to 100 h, 5 to 50 h, or 5 to 20 h. In particular, the Cu²⁺ ions are produced after at least 10 h or 5 h. In embodiments in which the catalyst is Pd/C, the catalyst may be activated prior to use at a temperature of 30 to 110° C. and a hydrogen flow rate of 60 L/h

The catalyst may be used at a concentration ranging from about 0.01 mmol to about 1 mmol. In certain embodiments, the catalyst may be at a concentration in a range of from 0.01 g/L to 100 g/L, preferably 0.1 g/L to 50 g/L, preferably 1 g/L to 30 g/L. In certain embodiments, the catalyst may be at a molar amount ranging from about 0.01 mmol to about 1 mmol, preferably 0.1 mmol to about 1 mmol, preferably 0.5 mmol to about 1 mmol. As should be appreciated, the catalyst may be recycled for one or more cycles during operation of the system (e.g., the system 100, 200, 300). Accordingly, in certain embodiments, the method disclosed herein includes recycling the catalyst for at least 1 to 15 cycles, preferably 1 to 12 cycles, 1 to 10 cycles, or 1 to 5 cycles. For example, the catalyst may be recycled for at least 2 cycles, 5 cycles, 10 cycles, 12 cycles, or 15 cycles with a conversion of bicarbonate to formate of at least 50 to 99%, 70 to 99%, 80 to 99%, 85 to 95%, 90 to 95%, 95% in every cycle

In certain embodiment, the method disclosed herein includes a cleaning step after the hydrogenation of bicarbonate to formate. For example, the cleaning step may remove at least divalent ions (e.g., Cu²⁺) produced in the hydrogenation reactor. In particular, the cleaning step is performed when using a copper catalyst in the hydrogenation reactor to convert the bicarbonate into formate.

The disclosed method may also include a step of passing the formate (HCOOM, where M is Na or K) produced by the hydrogenation reactor through an ion exchange resin reactor (e.g., the ion exchange resin reactor 130) to produce formic acid. The ion exchange resin reactor may form the formic acid from the HCOOM starting material in a yield ranging from about 1% to about 75% based on a molar amount of the formate. For example, the ion exchange resin reactor may form the formic acid in a yield of about 5%, about 10%, about 15%, about 20%, about 25%, about 30%, about 35%, about 40%, about 45%, or about 50%, where about includes plus or minus 2.5%, based on the amount starting material coming from the HCOOM.

In addition to forming formic acid by protonating formate, the ion exchange resin reactor also removes divalent ions (e.g., Cu²⁺) that may be present in the formate feed from the hydrogenation reactor. Therefore, in certain embodiments, the ion exchange resin reactor may also be used to perform the cleaning step discussed above. However, as should be appreciated, the cleaning step may be performed upstream of the ion exchange resin reactor using other cleaning techniques suitable for removing divalent ions.

The method also includes a step of capturing the formic acid formed by the ion exchange resin reactor in a HCOOH tank (e.g., the HCOOH tank 135). In some embodiments, the CO₂ may be used for back protonation of the ion exchange resin contained within the ion exchange reactor to regenerate the ion exchange resin.

The method further includes a step of producing at least a portion of a hydrogen gas used by the hydrogenation reactor, the Cl₂, and the base (MOH, where M is Na or K) by electrolysis of water in a chlorine-alkali electrolysis reactor (e.g., the chlorine-alkali electrolysis reactor 120). The chlorine-alkali electrolysis reactor may generate from about 1% to about 100% of the H₂ used by the hydrogenation reactor. For example, the chlorine-alkali electrolysis reactor may generate about 1%, about 10%, about 20%, about 30%, about 40%, about 50%, about 60%, about 70%, about 80%, about 90%, or about 100% of the H₂ used by the hydrogenation reactor, where about includes plus or minus 5%. In some embodiments, the method includes a step of transferring the MOH produced by the chlorine-alkali electrolysis reactor to the bubble column reactor through a MOH transfer line. Additionally, the method may include transferring the Cl₂ generated by the chlorine-alkali electrolysis reactor to the catalytic converter through the Cl₂ transfer line so that it may be combined with water to form HCl.

The method also includes a step of producing at least a portion of the hydrogen gas used by the hydrogenation reactor via electrolysis of water in a water electrolysis reactor (e.g., the water electrolysis reactor 125). The water electrolysis reactor may generate from about 1% to about 100% of the H₂ used by the hydrogenation reactor. For example, the water electrolysis reactor may generate about 1%, about 10%, about 20%, about 30%, about 40%, about 50%, about 60%, about 70%, about 80%, about 90%, or about 100% of the H₂ used by the hydrogenation reactor, where about includes plus or minus 5%.

According to some embodiments, the method may include a step of combining the formate (HCOOM) produced by the hydrogenation reactor with HCl in an esterification reactor (e.g., the esterification reactor 245) to produce a metal chloride (e.g., MCl, where M is Na or K) and methyl formate (HCO₂CH₃). For example, while in the esterification reactor, the formate undergoes a first protonation in the presence of a solvent such as, for example, water, and HCl (e.g., HCl from the chloro-alkali electrolysis reactor 120, the convertor 140, or both). The HCl may be used to adjust a pH within the esterification reactor. For example, the HCl may adjust the pH to a value ranging from 1 to 5, preferably 1 to 4, or 2. The first protonation in the esterification reactor results in an ester (HCO₂CH₃) that is fed to a hydrolysation reactor and distilled to generate a water-soluble carboxylic acid such as formic acid.

Accordingly, the method may also include a step of hydrolyzing the HCO₂CH₃ produced by the esterification reactor in a hydrolysis and distillation reactor (e.g., the hydrolysis and distillation reactor 250) to produce formic acid, and a step of capturing the formic acid formed by a hydrolysis and distillation reactor in the HCOOH tank. The hydrolyzing step (e.g., second protonating step) may be done in the presence of a solvent such as water and at a temperature ranging from between about 20 to 105° C., preferably between about 25 to 100° C., and preferably between about 80 to 100° C.

In some embodiments, the esterification of a formate solution may be carried out by adding a suitable HCl solution to achieve at least the stoichiometric amount of the included formate and a suitable excess (of HCl) to adjust acidic conditions. A mono-alcohol (e.g., MeOH/EtOH or any other suitable alcohol) is added and low boiling esters (e.g., having a boiling point less than 300° C.) that may form are removed (and optionally traces of unconverted alcohol and water are removed as well), preferably removal is achieved with heating. The alcohol and traces of unconverted ester may be recycled back to the esterification reactor 245 after distillation

In one embodiment, the method includes feeding the formate from the hydrogenation reactor (e.g., the hydrogenation reactor 115) to a reactor (e.g., the reactor 306) Certain embodiments of the disclosed method include a step of combining Cl₂ produced by the chloro-alkali electrolysis reactor with water to produce HCl in the catalytic converter and transferring the HCl generated in the catalytic converter to the ion exchange resin reactor through the HCl transfer line.

The present disclosure also includes embodiments of a method for generating a formic acid precursor having formate (HCOOM, where M is Na or K) from a CO₂ gas stream (e.g., the CO2 gas stream 105) using the system of FIGS. 1 and 2. The method includes the step of (a) passing the CO₂ gas stream through a bubble column disposed in a bubble column reactor (e.g., the bubble column reactor 110) in which the bubble column includes at least one base (MOH, where M is Na or K). While in the bubble column, the CO₂ in the CO₂ gas stream is combined or otherwise mixed with the base to produce bicarbonate (MHCO₃) and an off gas. For example, CO₂ in the CO₂ gas stream reacts with the base (e.g., NaOH or KOH) in the bubble column to generate the respective bicarbonate (e.g., NaHCO₃ or KHCO₃). In one embodiment, the base used is KOH. In another embodiment, the base used is NaOH. In other embodiments, both KOH and NaOH are used to generate the bicarbonate compound(s). Following formation of the bicarbonate compound(s), the also includes a step of (b) combining the MHCO₃ with hydrogen gas in a hydrogenation reactor (e.g., the hydrogenation reactor 115) disposed downstream from and fluidly coupled to the bubble column reactor. The hydrogenation reactor receives the bicarbonate compound(s) from the bubble column where the bicarbonate compound(s) is mixed reacts with the hydrogen gas at a temperature of up to 210° C., and a hydrogen pressure ranging from about 0.001 bara to about 100 bara to produce formate (HCOOM, where M is Na or K). The hydrogen gas may be, preferably, at a temperature in the range of from about 15° C. to about 75° C., about 15° C. to about 150° C., or about 15° C. to about 210° C. In certain embodiments, the hydrogenation reactor includes at least one hydrogenation catalyst (e.g., a palladium (Pd) catalyst, a platinum (Pt) catalyst, and/or a nickel (Ni) catalyst) that facilitates formation of formate from the bicarbonate compound(s) and hydrogen gas.

The method also includes step (c) passing the HCOOM through a protonated ion exchange resin reactor (e.g., the ion exchange resin reactor 130) to produce formic acid. For example, the protonated ion exchange reactor is disposed downstream from and fluidly coupled to the hydrogenation reactor such that the ion exchange resin reactor receives the formate generated in the hydrogenation reactor where the Na and/or K metal is exchanged for the proton (H⁺) in the ion exchange resin to form formic acid (HCOOH). In addition, when using a copper catalyst, the ion exchange resin reactor may be used to remove divalent ions (e.g., Cu²⁺) that may form in the hydrogenation reactor. In certain embodiments, the ion exchange resin reactor includes a cation exchange resin. The ion exchange resin may be regenerated by flowing a stream of hydrochloric acid (HCl) provided by a convertor (e.g., the catalytic convertor 140) to release the Na and/or K from the ion exchange resin by ion exchange with H⁺, thereby forming a metal halide (MCl, where M is Na or K). In certain embodiments the method further includes step (d) electrolyzing an aqueous solution of the metal halide (e.g., potassium chloride (KCl) or sodium chloride (NaCl)) in a chlorine-alkali electrolysis reactor (e.g., the chlorine-alkali electrolysis reactor 120) disposed downstream from and fluidly coupled to the ion exchange resin reactor to produce at least one of: a portion of the hydrogen gas used in step (b), Cl₂, and a portion of the MOH used in step (a). The system used to perform the acts of the methods is arranged in a manner such that the chloro-alkali electrolysis reactor provides (i) at least a portion of the generated hydrogen gas to the hydrogenation reactor, (ii) at least a portion of the MOH to the bubble reactor column, and (iii) at least a portion of the generated Cl₂ to the catalytic convertor.

In an alternative embodiment, the method does not use an ion exchange resin reactor to generate the formic acid. Rather, the method uses an esterification reactor (e.g., the esterification reactor 245). Accordingly, in this particular embodiment, the method includes steps (e) combining the HCOOM produced by the hydrogenation reactor with HCl in the esterification reactor to produce the metal halide (MCl, where M is Na or K) and an ester (e.g. methyl formate (HCO₂CH₃) or ethyl formate (HCO₂C₂H₅)), (f) hydrolyzing the ester produced in the esterification reactor in a hydrolysis and distillation reactor (e.g., the hydrolysis and distillation reactor 255) to produce formic acid, and (g) electrolyzing water in the chlorine-alkali electrolysis reactor to produce at least one of: a portion of the hydrogen gas in step (b), Cl₂, and at least a portion of the MOH in step (a). In this particular embodiment, the chlorine-alkali electrolysis reactor receives the metal halide (MCl) from the esterification reactor to produce the Cl₂ and the MOH. In certain embodiments, the MCl from the esterification reactor is purified in a salt purifier disposed between and fluidly coupled to the esterification reactor and the chlorine-alkali electrolysis reactor.

### EXAMPLES

The following illustrate some specific example embodiments of the present disclosure. As should be appreciated by those of skill in the art, changes to the disclosed embodiments may be made in without departing from the spirit and scope of the application.

### Example 1

Hydrogenation of Bicarbonate

The following two methods (inside an autoclave (a) and a loop reactor (b)) are examples of a process for hydrogenating bicarbonate.

### a) Autoclave

### Activation of Catalyst:

5 g of mm-sized particles of the oxidic catalyst Pd on carbon catalyst are installed inside a rotating cage, which is placed inside a 250 mL autoclave system. The reactor is closed and flushed with nitrogen for 60 min (flow rate=60 L/h at ambient temperature—thereafter hydrogen is added stepwise to the stream replacing the nitrogen completely (hydrogen flow rate=60 L/h). The reactor is heated up to 100° C. (5K/min). That temperature is maintained for 1 h—followed by a cooling step down to the required reaction temperature of 30-80° C. The hydrogen flow is reduced to 10 L/h.

### Conversion/Hydrogenation Sequence:

The potassium bicarbonate solution (100 mL, 1-3 M) is put inside the reactor by use of a common HPLC pump. The stirrer starts at 300 rpm and the reactor valves are closed to adjust an overall reactor pressure of up to 30 bara by constant addition of hydrogen via pressure control. Samples are taken frequently by use of a sampling unit, which can be applied without changing the reaction parameters. The created potassium formate is analysed by ion chromatography.

The following conversion rates are detected after 6 and 24 hrs:

### Catalyst: 5 wt. % Pd/C

Pressure: 30 bara

### Temperature: 35° C.

Conversion: 6 h=97%
24 h=98%

After 24 hrs the reaction mixture is removed completely out of the reactor by use of an uptake pipe. Hydrogen supply is adjusted at 10 L/h after the removal of the liquid and the hydrogenation sequence can be repeated as described above.

The catalyst performance decreases over time, for example after the 12th cycle

### Catalyst: 5 wt. % Pd/C

Pressure: 30 bara

### Temperature: 35° C.

Conversion: 6 h=68%
24 h=90%
   The catalyst can be regenerated as follows:

The catalyst is washed with 2×200 mL water and heated up to 120° C. (5K/min) under nitrogen flow (60 L/h). 1 vol. % oxygen is added for 60 min. The oxygen addition is stopped, and the catalyst is treated with nitrogen for 2 h before 5 vol. % hydrogen is added for 16 hrs.

The catalyst performance can be recovered after 12 cycles, to:

### Catalyst: 5 wt. % Pd/C

Pressure: 30 bara

### Temperature: 35° C.

Conversion: 6 h=90%
24 h=94%

### b) Loop Reactor System

Inside a loop reactor system, the hydrogenation of potassium bicarbonate is carried out at temperatures <80° C. and an overall pressure of 30 bara. A volume of 500 mL mm-sized oxidic Pd/C catalyst (5 wt. % Pd) are place inside a tubular reactor (ID ˜ 2 inch). The button and the head of the reactor are connected with a liquid cycle line. Two pumps are installed to maintain a liquid cycle flow and to add new feed to the system. A second cycle line provides a gas flow from a top space of the reactor to the button position, which is connected to a gas supply to maintain the overall pressure. The gas is added directly inside the liquid reservoir to saturate the water in the liquid reservoir.

On top of the catalyst bed, 200 mL inert mm-sized ceramic or steel spheres are placed to allow mixing of the gas and liquid phase while passing the gas through the catalyst bed, which enter the reactor at the top position of the reactor.

### Activation of Catalyst:

500 g of mm-sized particles of the oxidic catalyst Pd on carbon catalyst are installed inside the tubular reactor. A top bed of the spheres is placed, after which the reactor is closed, and the bed is flushed up-flow with nitrogen for 60 min (flow rate=500 L/h at ambient temperature—thereafter hydrogen is added stepwise to the stream replacing the nitrogen completely (hydrogen flow rate=500 L/h). The reactor was heated up to 100° C. (5K/min). That temperature is maintained for 1 h—followed by a cooling step down to the required reaction temperature of 30-80° C. The hydrogen flow is reduced to 10 L/h.

### Conversion/Hydrogenation Sequence:

The potassium bicarbonate solution (1-3 M) is put inside the reactor by use of a common HPLC pump. After reaching a desired volume of 2.5 L of potassium bicarbonate the addition is stopped. The liquid cycle (150 L/h) and, thereafter, the gas cycle (1000 L/h) are put in operation. The addition of hydrogen is started to build up and maintain the overall reaction pressure (30 bara).

Samples are taken frequently by use of a sampling unit, which can be applied without changing the reaction parameters. The created potassium formate is analysed by ion chromatography. The following conversion rates were detected after 6 and 24 hrs:

### Catalyst: 5 wt. % Pd/C

Pressure: 30 bara

### Temperature: 80° C.

Conversion: 6 h=80%
24 h=90%

### Example 2

### Ion Exchange Process

The ion exchange processes apply to both potassium and sodium. However, to facilitate discussion of this process, reference will only be made to potassium.

a) Treatment of a Potassium Formate—Generation of Formic Acid Solution in Water.

500 mL of 1 M solution of potassium formate are processed (down flow) with a flow rate of 1 mL/min over a protonated strong cationic exchanger (Dowex 50-WX8-Mesh 50-100). The ion exchanger (550 g) is placed inside a chromatographic column with an inner diameter of 30 mm. Samples are taken frequently at the outlet. The formed formic acid and the potassium content are analysed by ion chromatography.

At the described conditions the amount of the formic acid generated at the outlet corresponds to the amount of processed potassium formate minus the potential hold-up of the ion-exchanger bed (˜5% of the added formate). The potassium concentration in the final solution is below the detection limit.

b) Treatment of a Potassium Formate Containing Potassium Bicarbonate Solution—Generation of Formic Acid Solution in Water.

In a first step the carbon dioxide/bicarbonate is removed in a stirred vessel and in a second (no gas formation) step, the formation of formic acid, may be carried out in a chromatographic column. 500 mL of 1 M solution of potassium formate containing 0.15 mol bicarbonate is treated in a stirred vessel with an equimolar amount of ion exchanger to remove the potassium bicarbonate. The potassium is fixed on the ion exchanger and the carbon dioxide is liberated in the gas phase. The ion exchanger is removed by filtration.

After that the solution is pumped with a flow rate of 1 mL/min over a protonated strong cationic exchanger (Mesh 50-100). The cationic exchanger (amount corresponding to the amount of potassium, 470 g) is placed inside a chromatographic column with an inner diameter of 30 mm. Samples are taken frequently at the outlet. The formed formic acid and the potassium content are analysed by ion chromatography.

At the described conditions the amount of the generated formic acid at the outlet corresponds to the amount of processed potassium formate minus the potential hold-up of the ion exchanger bed (˜5% of the added formate). The potassium concentration in the final solution is below the detection limit.

Alternatively, the treatment of potassium formate containing potassium bicarbonate solution, and generation of formic acid solution in water, can be accomplished in a single step. 500 mL of 1 M solution of potassium formate containing 0.15 mol bicarbonate is treated in a stirred vessel with 550 g of ion exchange resin to remove substantially all the potassium. The potassium is fixed on the ion exchange resin and the carbon dioxide is liberated in the gas phase.

The formed formic acid and the potassium content are analysed by ion chromatography.

The ion exchange resin is removed by filtration.

At the described conditions, the amount of the formic acid generated corresponds to the amount of processed potassium formate minus the potential hold-up of the ion exchange resin (˜5% of the added formate). The potassium concentration in the final solution is below the detection limit.

c) Treatment of a Potassium Formate by Use of Weak Cation Exchanger

500 mL of 1 M solution of potassium formate is processed (down flow) with a flow rate of 1 mL/min over a protonated weak cationic exchange resin (Purolite C107E-Mesh 50-100). The ion exchange resin (75 g) is placed inside a chromatographic column with an inner diameter of 30 mm. Samples are taken frequently at the outlet. The formed formic acid and the potassium content are analysed by ion chromatography.

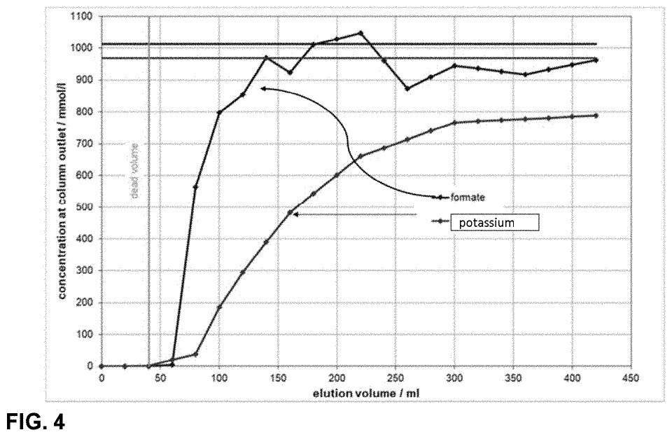

At the described conditions the amount of the formic acid generated at the outlet corresponds to the amount of processed potassium formate minus the potential hold-up of the ion exchanger bed (˜5% of the added formate). The potassium is removed out of the product only in part. For example, approx. 80% of the added potassium is still present in the product, as shown in FIG. 4. FIG. 4 is a plot of the concentration of formate and sodium at an outlet of the chromatographic column in millimoles (mmol)/liter (l) as a function of the elution volume in mL. The plot potassium removal from the above described sodium formate solution with a weak cationic exchange resin based on ion chromatography analysis of the water phase at the outlet of the column.

Regeneration of the cation exchange resin by hydrochloric acid may be done using any suitable regeneration technique known in the art that removes sodium from and protonates the cation exchange resin. For example, after the described loading processes, the cation exchange resin (loaded with potassium >50% of maximum loading) is re-protonated, i.e. transferred to the protonated form, by use of the following procedure. After the addition of 5 bed volume of deionized water with a flow rate of 10 mL/min, the ion exchange resin bed is treated with 5 bed volumes of 5 wt. % hydrochloric acid (3 mL/min). Finally, the bed is washed again with 5 bed volume of deionized water with a flow rate of 10 mL/min

e) Regeneration of Weak Cation Exchanger by Use of Carbon Dioxide

A potassium loaded ion exchanger (75 g Purolite C107E-Mesh 50-100) is placed in a pressure stable chromatographic column (ID=20 mm). At ambient temperature 10-30 bed volumes of carbon dioxide saturated deionized water are added from the top with flow rate of 2 mL/min at 30 bara. After the described treatment approx. 90% of the sodium are detected inside the eluate phase. The amount of rinsing water (bed volume) depends on the loading and the amount and type of functional groups of the ion exchanger. Complete regeneration is possible with 50 bed volumes.

The regeneration can be carried out at slight overpressure as well as at higher pressure (pCO₂=2-50 bara). At higher pressures the regeneration is more effective because the solubility of carbon dioxide is higher.

### Example 3

### Generation of Chlorinated Water

At room temperature (20° C.), a Cl₂ stream (<10 ml/min range) is inserted finely distributed at the bottom position to a chromatographic/bubble column, which is filled with 2 L water. Non-adsorbed Cl₂ leaves the column at the top position of the column. After a concentration >1 g CL2/L is achieved inside the liquid phase. The liquid contains solved chlorine, chloride, hypochlorite, and chlorate. (After removing of hypochlorite and/or chlorate) the liquid can be applied to regenerate a cation exchanger according example (see above—“Regeneration of cation exchanger by hydrochloric acid”).

The removal of hypochlorite and/or chlorate should be inserted inside the embodiment of the patent application. Removal can be carried out by chemical reduction and/or adsorption at suitable adsorbents.

### CO₂ Capture

At room temperature (20° C.) and ambient pressure (1 bara), a CO₂ containing stream of N₂ (and O₂) is inserted at the bottom position to a chromatographic/bubble column, which is filled with 2 L water and 4.6 mol KOH (2.3 mol KOH/L). The gas stream leaves the column at the top position of the column. A couple of experiments is carried out at overpressure of 1 bara, which was adjusted at the top of the column by a suitable outlet vent and a connected manometer. Gas flow is measured and adjusted in all cases at the inlet line. After 48 hrs of operation the following concentrations are determined inside the solution by TIC (total inorganic carbon) detection.

TABLE 1

| | C | | Overpressure | Sum gas | |
|---|---|---|---|---|---|
| | CO2, | | Outlet, | flow, | C HCO3—, |
| # | vol. % | Medium | bara | L/min | mol/L |
| 1 | 5 | N₂ | ~0.05 | 2 | 2.10 |
| 2 | 15 | Air | ~0.05 | 2 | 2.28 |
| 3 | 50 | N₂ | ~0.05 | 2 | 2.30 |
| 4 | 50 | N₂ | ~1.00 | 2 | 2.30 |

### Example 4

Thermal processing of a formate/bicarbonate mixtures inside a batch distillation. To generate the ester a solution of 8.2 kg water, 1.69 kg potassium chloride (KCl), 0.88 kg formic acid (HCOOH) is mixed with 1.84 kg of methanol. The mixture is stirred and suitable amount of concentrated HCl (20-30 wt. %) is added to adjust a pH value of ˜2, which can be determined with suitable pH measurement equipment. During the process the in-situ formed ester is distilled off. For example, a distillation unit includes a heating coil upstream from a first distillation column, and a second distillation column fluidly coupled to the first distillation column. Methanol, water, formic acid, a metal halide (MCl) and HCl are feed into the heat exchanger, whereby methyl formate is generated and distilled over a top of the first distillation column. The methyl formate and water is fed to the second distillation column and hydrolysed into the corresponding products (e.g., methanol and formic acid). Therefore 4.8 kg water are filled in the second distillation column at the beginning of each batch. The methanol exits through a top of the second distillation column and may be fed to the heating coil after distillation. The distillation unit is at a pressure of about 1 bara. The distillation columns used include a packed bed, RASCHIG rings, isolated, 25 theoretical plates: ID˜5 cm, Height˜100 cm. A temperature of the heating coil and at a bottom of the second distillation column is >70° C.

At the following mass balance can be determined as depicted in Table 2.

TABLE 2

| Compound, | | | | K-2 out |
|---|---|---|---|---|
| kg/h | Educt | R-1 out | K-2 in | Bottom |
| KCl | 1.69 | 1.69 | | |
| Water | 8.20 | 8.48 | 4.80 | 3.72 |
| HCOOH | 0.88 | 0.18 | | 0.56 |
| Methanol | 1.84 | 1.19 | | |
| Methyl formate | 0 | 0 | | |

For the present Examples, analysis by ion chromatography the following hardware and conditions were applied:

### Ion Chromatography Anions

instrument: DIONEX ICS 1000 ion chromatography system
separation column: Metrosep A 7, 250 mm×4 mm
column pressure: 11 MPa
suppressor: ASRS-I, 4 mm
temperature: 25° C.
eluent: 3.6 mmol/l sodium carbonate/water isocratic
eluent flow: 0.7 ml/min
injection volume: 25 μl
detector: Conductivity detector
retention time: hydroxy acetic acid (6.26 min), acetic acid (6.48 min), formic acid (7.26 min), fluoride (5.95 min), chloride (9.51 min), nitrite (11.59 min), bromide (14.97 min), nitrate (17.22 min), phosphate (21.68 min), sulfate (25.57 min)

For the termination of total organic compounds, the following hardware and conditions were applied:

### Determination of TC, TIC and TOC

instrument: multi N/C 3100 Analytik Jena AG
oxygen flow: 9.6 l/h
detector: infrared detector for carbon dioxide
   injection volume: 200 μl in Quartz reactor, 800° C. (TC)
   injection volume: 200 μl in 10% phosphoric acid (TIC)

Persons skilled in the art may make various changes in the shape, size, number, separation characteristic, and/or arrangement of parts without departing from the scope of the instant disclosure. Each disclosed component, system, and process step may be performed in association with any other disclosed component, system, or process step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. Where desired, some embodiments of the disclosure may be practiced to the exclusion of other embodiments.

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value+/−about 10%, depicted value+/−about 50%, depicted value+/−about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

1. A method for generating a carboxylic acid from carbon dioxide (CO₂), the method comprising:
(a) feeding a gas stream comprising the CO₂ to a first reactor comprising a base (MOH) to produce bicarbonate (MHCO₃);
(b) feeding the MHCO₃ generated in the first reactor to a second reactor disposed downstream from the first reactor, wherein the second reactor comprises a catalyst;
(c) contacting the MHCO₃ with hydrogen gas in the presence of the catalyst in the second reactor to produce formate (HCOOM); and
(d) electrolysing an aqueous solution of a metal halide (MCl) in a chloro-alkali electrolysis reactor fluidly coupled to the first reactor, the second reactor, or both to produce at least a portion of the MOH, the hydrogen gas and Cl₂, wherein the portion of the MOH is used in step (a), and wherein the carboxylic acid is formic acid (HCOOH).

2. The method of claim 1, comprising feeding the hydrogen gas and the Cl₂ produced in the chloro-alkali electrolysis reactor to a converter disposed downstream from the chloro-alkali electrolysis reactor, wherein the converter is configured to produce hydrochloric acid (HCl).

3. The method of claim 2, further comprising (e) protonating the HCOOM to produce the HCOOH and, optionally, the MCl, wherein protonating comprises passing the HCOOM through an ion exchanger comprising an ion exchange resin configured to protonate the HCOOM to generate the HCOOH.

4. The method of claim 2, further comprising (e) protonating the HCOOM to produce the HCOOH and, optionally, the MCl, wherein protonating comprises combining the HCOOM with hydrochloric acid (HCl) in a distillation reactor, to produce the HCOOH and the MCl, wherein the convertor provides at least a portion of the HCl.

5. The method of claim 2, further comprising (e) protonating the HCOOM to produce the HCOOH and, optionally, the MCl, wherein protonating comprises combining, in a first protonating step, the HCOOM with a mono-alcohol in the presence of HCl to produce an ester and the MCl and, in a second protonating step, hydrolysing the ester to produce the formic acid, wherein the convertor provides at least a portion of the HCl.

6. The method of claim 5, wherein the mono-alcohol is methanol (CH₃OH) or ethanol (C₂H₅OH), wherein the ester is methyl formate (HCO₂CH₃) or ethyl formate (HCO₂C₂H₅), and wherein the MCl is KCl or NaCl.

7. The method of claim 5, recovering the HCOOH via distillation in a distillation reactor.

8. The method of claim 1, wherein the MOH is potassium hydroxide (KOH)) or sodium hydroxide (NaOH), and wherein a temperature and a hydrogen pressure within the second reactor is at or below 210° C. and in a range of from 0.001 bara to 100 bara, respectively.

9. The method of claim 1, wherein the catalyst comprises at least one catalyst selected from the group consisting of a copper catalyst, a palladium catalyst, a nickel catalyst, and a platinum catalyst.

10. The method of claim 1, wherein at least a portion of the HCOOM generated in step (b) is converted to oxalic acid (HOOC—COOH).

11. A system for generating a carboxylic acid from carbon dioxide (CO₂) comprising:
a first reactor fluidly coupled to a gas source comprising the CO₂, and configured to combine the CO₂ with a base (MOH) to generate bicarbonate (MHCO₃) and, optionally, an off gas;
a second reactor disposed downstream from and fluidly coupled to the first reactor and comprising a catalyst, wherein the second reactor is configured to receive the bicarbonate and hydrogen gas and to produce formate (HCOOM), and wherein a temperature and hydrogen pressure within the second reactor is in the range of from 15° C. to 210° C. and from 0.001 bara to 100 bara, respectively; and
a chloro-alkali electrolysis reactor disposed downstream from and fluidly coupled to the first reactor and the second reactor, wherein the chloro-alkali electrolysis reactor is configured to produce at least a portion of the base, a hydrogen gas and chlorine (Cl₂), and to provide at least a portion of the base to the first reactor.

12. The system of claim 11, further comprising an ion exchange resin reactor comprising a protonated cation exchange resin disposed downstream from and fluidly coupled to the second reactor and upstream from and fluidly coupled to the chloro-alkali electrolysis reactor, wherein the ion exchanger is configured to generate the carboxylic acid and, optionally, a metal halide, wherein the carboxylic acid is formic acid (HCOOH), oxalic acid (HOOC—COOH), or both.

13. The system of claim 11, further comprising a distillation reactor disposed downstream from and fluidly coupled to the second reactor and upstream from and fluidly coupled to the chloro-alkali electrolysis reactor, wherein the distillation reactor is configured to protonate the HCOOM with hydrochloric acid (HCl) to produce the carboxylic acid and a metal halide (MCl), wherein the carboxylic acid is HCOOH.

14. The system of claim 11, further comprising an esterification and hydrolysation reactor disposed downstream from and fluidly coupled to the second reactor and upstream from and fluidly coupled to the chloro-alkali electrolysis reactor, wherein the esterification and hydrolysation reactor is configured to receive the HCOOM and to generate the carboxylic acid and, optionally, a MCl, wherein the carboxylic acid is HCOOH.

15. The system of claim 14, comprising a distillation reactor disposed downstream from and fluidly coupled to the esterification and hydrolysation reactor, wherein the distillation reactor is configured to isolate the HCOOH.

16. The system of claim 11, comprising a converter disposed downstream from and fluidly coupled to the chloro-alkali electrolysis reactor and configured to receive the Cl₂ and the hydrogen gas from the chloro-alkali electrolysis reactor, to produce hydrochloric acid (HCl), and to provide the HCl to a third reactor fluidly coupled to the second reactor and the chloro-alkali electrolysis reactor, wherein the third reactor is an esterification and hydrolysation reactor, an ion exchange resin reactor, or a distillation reactor, and wherein the third reactor is configured to generate the carboxylic acid and, optionally, a MCl, and wherein the carboxylic acid is HCOOH.

17. The system of one of claim 11, wherein the catalyst is selected from the group consisting of a copper catalyst, a palladium catalyst, a nickel catalyst, and a platinum catalyst.

18. A method for generating a carboxylic acid from carbon dioxide (CO₂), comprising:
(a) mixing a gas stream comprising the CO₂ with a base (MOH) to produce bicarbonate (MHCO₃);
(b) contacting the MHCO₃ with hydrogen gas in the presence of the catalyst to produce formate (HCOOM); and
(c) electrolysing an aqueous solution of a metal halide (MCl) to produce at least a portion of the MOH used in step (a), wherein the carboxylic acid is formic acid (HCOOH).

19. The method of claim 18, comprising (e) protonating the HCOOM using hydrochloric acid (HCl) to generate the HCOOH, wherein a by-product of the protonation is MCl, wherein a portion of the MCl generated from the protonation of the HCOOM is used for the aqueous solution electrolyzed in step (d), and wherein the MCl is potassium chloride (KCl) or sodium chloride (NaCl).

20. The method of claim 18, wherein the protonating of the HCOOM comprises first protonating the HCOOM with a mono-alcohol in the presence of the HCl to generate an ester and the MCl followed by protonating the ester to generate the HCOOH, wherein the mono-alcohol is methanol (CH₃OH) or ethanol (CH₃CH₂OH).

21. The method of claim 18, wherein at least a portion of the HCOOM generated in step (b) is converted to oxalic acid (HOOC—COOH).
